(19) United States
(12) Patent Application Publication
(10) Pub. No.: US 2025/0248611 A1
(43) Pub. Date: Aug. 7, 2025

(54) INTRAVASCULAR OPTICAL DIFFUSE BLOOD FLOW CORRELATION SPECTROSCOPY

(71) Applicant: INFRAREDX, INC., Bedford, MA (US)

(72) Inventors: Cherry GREINER, Concord, MA (US); Craig GARDNER, Belmont, MA (US); Nicholas GIGLIO, Woburn, MA (US)

(73) Assignee: INFRAREDX, INC., Bedford, MA (US)

(21) Appl. No.: 18/431,832

(22) Filed: Feb. 2, 2024

(51) Int. Cl. A61B 5/026; A61B 5/00; A61B 5/02; A61B 5/0285

(52) U.S. Cl. CPC A61B 5/0261; A61B 5/02007; A61B 5/0285; A61B 5/6852; A61B 5/7278; A61B 2562/0233

(57) ABSTRACT

A method for evaluating characteristics of a blood vessel and/or of blood flowing within the blood vessel comprises (a) delivering light at one or more wavelengths into the blood vessel using a light delivery portion of a catheter, (b) capturing reflected light at each wavelengths and generating a signal corresponding to light captured at each wavelength using a light capturing portion of the catheter, and (c) determining blood flow rate and/or blood flow velocity within the blood vessel from the signal generated in step (b) by the light capturing portion of the catheter. The method may also include the step of (d) determining a wall sheer stress level and/or fractional flow reserve ratio for the blood vessel from the blood flow rate and/or blood flow velocity determined in step (c).

### FIELD OF THE DISCLOSURE

This disclosure relates to optical sensing of intravascular blood flow. In particular, this disclosure relates to calculating intravascular fractional flow reserve and wall sheer stress through direct measurements of optical diffuse blood flow by spectroscopy.

### BACKGROUND

Stenosis occur as a symptom of coronary artery disease where plaque builds up and blocks the normal flow of blood in an artery. Fractional flow reserve (FFR) is a physiology measurement used to characterize and assess the severity of a vascular stenosis and to guide clinical decisions related to stenting. FFR is the ratio of maximum achievable blood flow through a blockage (area of stenosis) to the maximum achievable blood flow in the same vessel in the hypothetical absence of the blockage. Traditionally, the severity of a stenosis may be measured using a mechanical pressure wire which indirectly measures blood flow. A clinician may place the wire in two positions within the blood vessel (one before and one after the stenosis) and the ratio of these pressure readings before and after the stenosis is converted into the FFR. A FFR lower than 0.75-0.80 is generally considered to be associated with myocardial ischemia.

Wall sheer stress is a measurement of the tangential force per unit area that flowing blood exerts on a blood vessel wall and is a critical identifier when determining plaque progression and blood vessel wall health. The initiation of plaque formation is strongly linked to an increase in wall sheer stress. For example, in healthy human coronary arteries, the wall shear stress may be approximately 1.4 Pa. In a coronary artery with stenosis, the wall shear stress may reach >7 Pa in some instances.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

Aspects of the disclosure are embodied in a method for evaluating characteristics of a blood vessel and/or of blood flowing within the blood vessel. The method includes (a) delivering light at one or more wavelengths into the blood vessel using a light delivery portion of a catheter. The method includes (b) capturing reflected light at each of the one or more wavelengths and generating a signal corresponding to light captured at each of the one or more wavelengths using a light capturing portion of the catheter. The method includes (c) determining at least one of blood flow rate and blood flow velocity within the blood vessel from the signal generated in step (b) by the light capturing portion of the catheter.

According to other aspects, the method includes (d) determining at least one of a wall sheer stress level and/or fractional flow reserve ratio for the blood vessel from the at least one of blood flow rate and blood flow velocity determined in step (c).

According to other aspects, the light delivery portion comprises a first optical guide extending along the catheter and the light capturing portion comprises a second optical guide extending along the catheter, and wherein the first optical guide is the same as or different than the second optical guide.

According to other aspects, the light delivery portion comprises an LED and/or laser coupled to the first optical guide and the light capturing portion comprises a photodetector coupled to the second optical guide.

According to other aspects, the light delivery portion comprises a light source located at an optical interface of the catheter, and the light capturing portion comprises a light detector located at the optical interface.

According to other aspects, step (c) comprises performing a time series analysis for the signal generated in step (b) by the light capturing portion of the catheter, wherein the time series analysis comprises an autocorrelation and/or autocovariance.

According to other aspects, the time series analysis is conducted for one or more lag times.

According to other aspects, step (c) comprises: obtaining one or more linear models based on the time series analysis, wherein the one or more linear models provide an estimated flow rate at a selected lag time; and inputting a result of the time series analysis for the captured reflected light at the selected lag time into the one or more linear models to determine a flow rate corresponding to the result of the time series analysis.

According to other aspects, each of the one or more linear models is associated with a different wavelength of light.

According to other aspects, step (d) comprises calculating the fraction flow reserve ratio by: obtaining a hypothetical blood flow velocity within the blood vessel; and comparing the calculated at least one of blood flow rate and blood flow velocity of the blood vessel with the hypothetical blood flow velocity.

According to other aspects, step (c) comprises determining the blood flow rate and step (d) comprises calculating the wall shear stress by the formula: τ=4 μQ/πr³, where: τ represents the wall shear stress; μ (mu) is the dynamic viscosity of blood; and Q is the blood flow rate determined in step (c).

According to other aspects, the one or more wavelengths include a first wavelength and a second wavelength, wherein the first wavelength is associated with a first penetration depth and the second wavelength is associated with a second penetration depth, the first penetration depth being less than the second penetration depth.

According to other aspects, the method includes switching between the first wavelength and the second wavelength by activating a switch.

Aspects of the disclosure are embodied in a system for evaluating characteristics of a blood vessel and/or of blood flowing within the blood vessel. The system includes an intravascular catheter with a light delivery portion and a light capture portion. The system includes a controller coupled to each of the light delivery portion and the light capture portion of the catheter. The controller is programmed to execute a procedure comprising: (a) delivering light at one or more wavelengths into the blood vessel using the light delivery portion of the catheter; (b) capturing reflected light at each of the one or more wavelengths and generating a signal corresponding to light captured at each of the one or more wavelengths using the light capturing portion of the catheter; and (c) determining at least one of blood flow rate and blood flow velocity within the blood vessel from the signal generated in step (b) by the light capturing portion of the catheter.

According to other aspects, the procedure further comprises: (d) determining at least one of a wall sheer stress level and/or fractional flow reserve ratio for the blood vessel from the at least one of blood flow rate and blood flow velocity determined in step (c).

According to other aspects, the light delivery portion comprises a first optical guide extending along the catheter and the light capturing portion comprises a second optical guide extending along the catheter, and wherein the first optical guide is the same as or different than the second optical guide.

According to other aspects, the light delivery portion comprises an LED and/or laser coupled to the first optical guide and the light capturing portion comprises a photodetector coupled to the second optical guide.

According to other aspects, the light delivery portion comprises a light source located at an optical interface of the catheter, and the light capturing portion comprises a light detector located at the optical interface.

According to other aspects, to perform step (c), the controller is programed to perform a time series analysis for the signal generated in step (b) by the light capturing portion of the catheter, wherein the time series analysis comprises an autocorrelation and/or autocovariance.

According to other aspects, the time series analysis is conducted for one or more lag times.

According to other aspects, to perform step (c), the controller is programmed to obtain one or more linear models based on the time series analysis, wherein the one or more generated linear models provide an estimated flow rate at a selected lag time; and to input a result of the time series analysis for the captured reflected light at the selected lag time into the one or more linear models to determine a flow rate corresponding to the result of the correlation analysis.

According to other aspects, each of the one or more linear models is associated with a different wavelength of light.

According to other aspects, to perform step (d), the controller is programmed to calculate the fraction flow reserve ratio by (1) obtaining a hypothetical blood flow velocity within the blood vessel; and (2) comparing the calculated at least one of blood flow rate and blood flow velocity blood flow velocity of the blood vessel with the hypothetical blood flow velocity.

According to other aspects, step (c) comprises determining the blood flow rate and step (d) comprises calculating the wall shear stress by the formula: τ=4 μQ/πr3, where: τ represents the wall shear stress; μ (mu) is the dynamic viscosity of blood; and Q is the blood flow rate determined in step (c).

According to other aspects, the one or more wavelengths include a first wavelength and a second wavelength, wherein the first wavelength is associated with a first penetration depth and the second wavelength is associated with a second penetration depth, the first penetration depth being less than the second penetration depth.

According to other aspects, the procedure includes switching between the first wavelength and the second wavelength by activating a switch.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 is a schematic cross-sectional view of a system for performing intravascular optical diffuse flow correlation spectroscopy;

FIG. 2 is a flow chart illustrating steps of a process for performing intravascular optical diffuse flow correlation spectroscopy;

FIG. 3 is a graph illustrating the mean autocorrelation at different known flow rates using near infrared spectroscopy (“NIRS”).

FIG. 4 is a graph illustrating the mean autocorrelation of FIG. 3 at a top lag.

FIG. 5 is a graph illustrating the mean autocorrelation at a selected top lag and the linear regression fit.

FIG. 6 is a graph illustrating the mean signal at different flow rates.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

### Definitions

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

References in the specification to “one embodiment,” “an embodiment,” a “further embodiment,” “an example,” “an exemplary embodiment,” “some aspects,” “a further aspect,” “aspects,” etc., indicate that the embodiment, example, or aspect described may include a particular feature, structure, or characteristic, but every embodiment encompassed by this disclosure may not necessarily include the particular feature, structure, or characteristic. Moreover, such phrases are not necessarily referring to the same embodiment, example, or aspect. Further, when a particular feature, structure, or characteristic is described in connection with an embodiment, such feature, structure, or characteristic is also a description in connection with other embodiments, examples, or aspects, whether or not explicitly described.

This description may use various terms describing relative spatial arrangements and/or orientations or directions in describing the position and/or orientation of a component, apparatus, location, feature, or a portion thereof or direction of movement, force, or other dynamic action. Unless specifically stated, or otherwise dictated by the context of the description, such terms, including, without limitation, top, bottom, above, below, under, on top of, upper, lower, left, right, in front of, behind, beneath, next to, adjacent, between, horizontal, vertical, diagonal, longitudinal, transverse, radial, axial, clockwise, counter-clockwise, etc., are used for convenience in referring to such component, apparatus, location, feature, or a portion thereof or movement, force, or other dynamic action represented in the drawings and are not intended to be limiting.

Unless otherwise indicated, or the context suggests otherwise, terms used herein to describe a physical and/or spatial relationship between a first component, structure, or portion thereof and a second component, structure, or portion thereof, such as, attached, connected, fixed, joined, linked, coupled, or similar terms or variations of such terms, shall encompass both a direct relationship in which the first component, structure, or portion thereof is in direct contact with the second component, structure, or portion thereof or there are one or more intervening components, structures, or portions thereof between the first component, structure, or portion thereof and the second component, structure, or portion thereof.

Unless otherwise stated, any specific dimensions mentioned in this description are merely representative of an exemplary implementation of a device embodying aspects of the disclosure and are not intended to be limiting.

To the extent used herein, the terms “about” or “approximately” apply to all numeric values and terms indicating specific physical orientations or relationships such as horizontal, vertical, parallel, perpendicular, concentric, or similar terms, specified herein, whether or not explicitly indicated. This term generally refers to a range of numbers, orientations, and relationships that one of ordinary skill in the art would consider as a reasonable amount of deviation to the recited numeric values, orientations, and relationships (i.e., having the equivalent function or result) in the context of the present disclosure. For example, and not intended to be limiting, this term can be construed as including a deviation of ±10 percent of the given numeric value, orientation, or relationship, provided such a deviation does not alter the end function or result of the stated value, orientation, or relationship. Therefore, under some circumstances as would be appreciated by one of ordinary skill in the art a value of about or approximately 1% can be construed to be a range from 0.9% to 1.1%.

To the extent used herein, the term “adjacent” refers to being near (spatial proximity) or adjoining. Adjacent objects or portions thereof can be spaced apart from one another or can be in actual or direct contact with one another. In some instances, adjacent objects or portions thereof can be coupled to one another or can be formed integrally with one another.

To the extent used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as stated as well as instances in which the event, circumstance, characteristic, or property occurs to a close approximation, such as accounting for typical tolerance levels or variability of the embodiments described herein.

To the extent used herein, the terms “optional” and “optionally” or the term “may” (e.g., as in the phrase “may include,” “may comprise,” “may produce,” “may provide,” or similar phrases) mean that the subsequently described, component, structure, element, event, circumstance, characteristic, property, etc. may or may not be included or occur and that the description includes instances where the component, structure, element, event, circumstance, characteristic, property, etc. is included or occurs and instances in which it is not or does not.

The term “diffuse correlation spectroscopy” refers to an optical modality that enables intravascular measurements of blood flow in deep tissue by quantifying the temporal light intensity fluctuations generated by dynamic scattering of moving red blood cells.

To the extent used herein, the terms “first” and “second” preceding the name of an element (e.g., a component, apparatus, location, feature, or a portion thereof or a direction of movement, force, or other dynamic action) are used for identification purposes to distinguish between similar elements, and are not intended to necessarily imply order, nor are the terms “first” and “second” intended to preclude the inclusion of additional similar elements. Furthermore, use of the term “first” preceding the name of an element (e.g., a component, apparatus, location, feature, or a portion thereof or a direction of movement, force, or other dynamic action) does not necessarily imply or require that there be additional, e.g., “second,” “third,” etc., such element(s).

To the extent used herein, the terms or phrases “configured to,” “adapted to,” “operable to,” “constructed and arranged to,” and similar terms mean that the object of the term or phrase includes, constitutes, or otherwise encompasses the requisite structure(s), mechanism(s), arrangement(s), component(s), material(s), algorithm(s), circuit(s), programming, etc. to perform a specified task or tasks or achieve a specified output or characteristic, either automatically or perpetually or selectively when called upon to do so.

### DESCRIPTION OF EMBODIMENTS

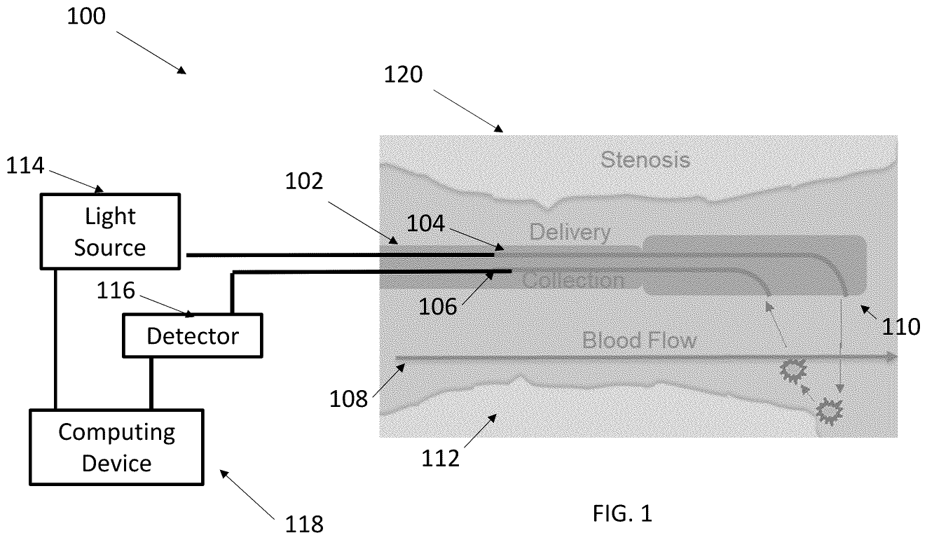

FIG. 1 is a schematic cross-sectional view of system 100 performing intravascular optical diffuse flow correlation spectroscopy. A patient may have an increased risk of heart disease as plaque 112 builds up in a blood vessel 120. To diagnose and assess cardiac conditions, a medical provider may elect to measure blood flow within the blood vessel 120 using a catheter 102.

The catheter 102 may be embodied as a stationary or rotating dual fiber intravascular catheter including a light delivery portion and a light collecting portion. In one example, the light delivery portion includes a delivery single mode fiber 104, and the light collection portion includes a collection multi-mode fiber 106. In some embodiments, the delivery fiber 104 comprises a single mode optical fiber configured to deliver coherent light into the blood vessel 120. The single mode optical fiber may be configured to allow light to travel in a “single path” down the center of its core, i.e., in the first or fundamental mode. In some applications, a single mode optical fiber experiences less signal dispersion and a higher bandwidth when compared to multi-mode fibers, although, in other examples, delivery fiber 104 may comprise a multi-mode fiber. The single mode optical fiber may be configured to allow for the transmission of a single mode of light or ray of light.

In some embodiments, the collection fiber 106 may be embodied as a multimode optical fiber separate from the delivery mode fiber 104 and may be configured to collect reflected light from the blood vessel 120. Each light ray collected by the multimode optical fiber may be at a marginally different reflection angle inside the fiber's core, and the multimode optical fiber is configured to collect multiple rays of light impacting it at different angles. The delivery fiber 104 and the collection fiber 106 may be oriented within the catheter 102 such that they are generally parallel to a blood flow direction 108. In some embodiments, the multimode optical fiber may have a larger core than a single mode fiber. The number of modes (M) that a multimode optical fiber may propagate may be approximated using the V-number which is determined by the radius of its core, the wavelengths, and the numerical aperture of the fiber.

Light source 114 may be coupled to a controller (e.g., a computing device 118) for controlling operation of the light source.

A single wavelength or multiple wavelengths of coherent or incoherent light may be combined and delivered by the delivery mode fiber 104. The light within the delivery mode fiber 104 may be directed by a direct fiber, a mirror, and/or a prism, so the light exits a side 110 of the catheter 102 (“side” illumination), transverse to the blood flow direction 108. In some embodiments, the delivery mode fiber 104 may be coupled to a light source 114 (e.g., laser). The light may be scattered by flowing red blood cells and reflected light may be collected by the collection fiber 106. The collection fiber 106 may collect light directly, using a mirror, and/or a prism and deliver light to a detector 116. In some embodiments, the collection fiber 106 may be coupled to the detector 116. Detector 116 generates a signal indicative of the intensity of reflected light that reaches the detector and may comprise a photodetector, such as a single-photon avalanche detector, a photodiode, photoelectric device, or any indium gallium arsenide (InGaAs) or a lead sulfide (PbS) detector.

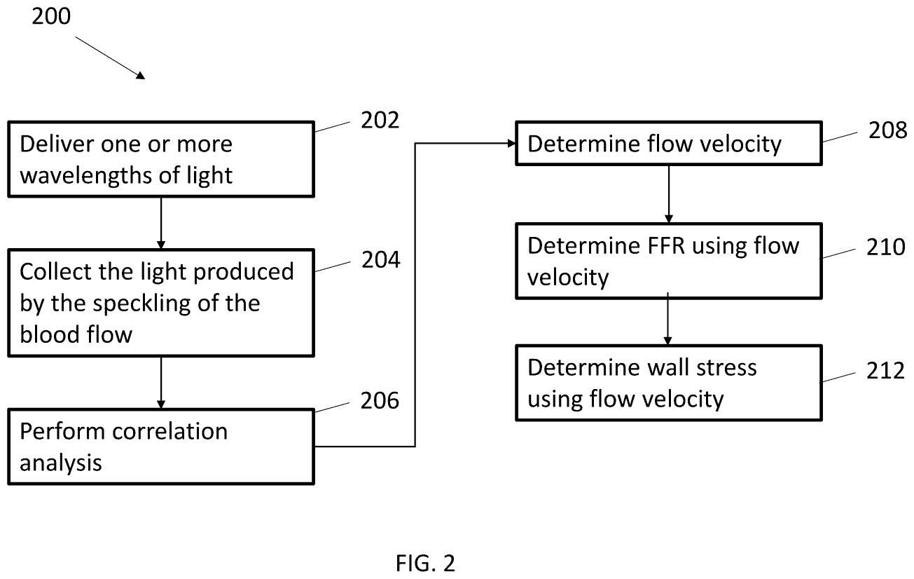

Computing device 118 may be coupled to the detector 116 and may be configured to calculate FFR and wall shear stress from the collected reflected light. A process performed by the computing device 118 to calculate FFR and wall shear stress is described in detail below in FIG. 2. The process may include analyzing the collected light using a time series analysis method (e.g., autocorrelation and/or autocovariance). In some embodiments, the computing device 118 may receive information associated with the collected light from another computing device. The process may include calculating blood flow velocity near and far from the blood vessel wall using the time series analysis method. The process may include calculating FFR and wall shear stress based on the blood flow velocity calculations and the velocity distribution across the blood vessel, which may be used as a blood vessel wall health metric and potentially guide clinical decisions.

Light source 114 and detector 116 may be coupled to the same controller (computing device) or to separate controllers.

Exemplary catheters and related systems for intravascular imaging and other intravascular optical processes, such as infrared spectroscopy, are described in U.S. Pat. Nos. 7,486,985; 7,539,530; 7,873,406; 8,052,605; 8,060,187; 8,280,495, and 8,386,023.

In some embodiments, the light delivery and light collection portions of the catheter 102 may include microelectronics located on the optical interface 110. The microelectronics of the light delivery portion may include one or more light sources (e.g., LEDs or laser) (not shown), and the microelectronics of the light collection portion may include one or more detectors (not shown). The one or more light sources may be configured to transmit light into the blood vessel 120, and the one or more detectors (e.g., reflective sensing photodiode or other photodetector) may be configured to collect the reflected light and generate a signal indicative of the intensity of the reflected light. In such embodiments, the delivery fiber 104 and the collection fiber 106 may be omitted from the catheter 102. Instead, the catheter 102 may include wiring (not shown) coupling the microelectronics on the catheter to device control system (e.g., computing device 118) for generating delivery light signals and for processing collected reflections signals, for example by method 200 described below.

System 100 provides a mechanism for delivering light into a blood vessel and collecting and measuring light reflected by blood within the blood vessel and from the blood vessel wall. Data signals generated by the system 100 may be used as described herein to determine blood flow rate (flow velocity) within the vessel, and the determined blood flow rate can be used to determine fractional flow reserve (FFR) and/or wall shear stress.

FIG. 2 shows a flow diagram illustrating one exemplary embodiment of a method 200 for measuring health of a blood vessel using intravascular optical diffuse flow correlation spectroscopy. Method 200 may be performed with or used in conjunction with any of the computer systems, devices, mechanisms, elements, or components disclosed herein, among other devices. Method 200 may be coded and stored as a computer-executable control algorithm for controlling the operation(s) of one or more of the computer systems, devices, mechanisms, elements, or components disclosed herein, among other devices. In various embodiments, some of the method steps shown may be performed concurrently, in a different order than shown, or may be omitted. Additional method steps may also be performed as desired. Flow begins at step 202.

At step 202, the catheter 102 may deliver one or more wavelengths of light into the blood vessel 100 using the delivery mode fiber 104. The one or more wavelengths may be provided by the light source 114 (e.g., laser) coupled to the delivery mode fiber 104. In some embodiments, the one or more wavelengths may include at least two different wavelengths of light to sample different portions of a velocity profile of the blood across the width of the blood vessel. The one or more wavelengths may be selected to have different optical properties through blood (e.g. scattering and absorption coefficients) so that the penetration depth of the light may be different depending on the wavelength. The depth resolution of blood flow may be improved/optimized through wavelength selection as some wavelengths will propagate deeper than others according to the Beer-Lambert law.

In some embodiments, light delivered in step 202 may include light at a first wavelength and light at a second wavelength. The first wavelength may be associated with a shallower blood penetration depth and may sample a smaller volume of blood near the wall of the catheter 102. The second wavelength may be associated with a deeper blood penetration depth and may sample a larger volume of blood, near to and farther away from the wall of the catheter 102, closer to the wall of the blood vessel 100. The first wavelength associated with the shallower penetration depth may be more suited for measurements in the vicinity of a tight lesion. In some embodiments, the first and second wavelengths may be continuous wave infrared light, where scattering dominates over absorption (wavelengths 1000 nm-2000 nm), with power from 1 mW to 50 mW.

In some embodiments, a switch (e.g., a virtual switch of a control algorithm executed a controller coupled to the light source 114) may be used to toggle between two coherent light sources emitting light at each of the two wavelengths for serial measurements or the light from a single light source may be split into the two wavelengths for simultaneous readings. The switch may also be used to select one of the wavelengths. Using more than one wavelength of light may allow for blood flow sampling in blood vessels with different lumen diameters and minimize reflective signal contribution from non-flowing artifacts (e.g., lumen wall).

At step 204, light reflected from flowing blood within the blood vessel 120 is collected in the collection fiber 106. The reflected light may be in the form of speckles, which are a physical phenomenon caused when the photons of light scatter from interacting with flowing red blood cells. The speckles may vary in time due to the flow of red blood cells as variations in blood flow velocity may change the diffuse speckle pattern. The variations in the scattered diffuse speckle pattern may be captured by the collection fiber 106 when the reflected light impacts the collection fiber 106.

Step 204 further includes transmitting the collected light to the detector 116 to generate a signal indicative of the intensity of the collected light contacting the detector 116.

At step 206, the computing device 118 coupled to the detector 116 performs a time series analysis of the collected light signal(s). The time series analysis may be embodied as a mathematical representation of a degree of similarity between a given time series and a lagged version of itself. In one example, the computing device 118 may calculate the autocorrelation and/or autocovariance of the collected light at sequential lags or time intervals.

At step 208, the computing device 118 determines the flow velocity of blood within the blood vessel 100 via a time series analysis method such as autocorrelation and/or autocovariance computed from the collected light. Flow velocity may be embodied as a mean square displacement from flowing interactions, which are modeled from Brownian motion, random flow models, and an autocorrelation function. In some embodiments, the computing device 118 may determine the flow velocity using the rate of change in the speckle signal intensity (autocorrelation or autocovariance) generated in step 206 with known flow rate curves.

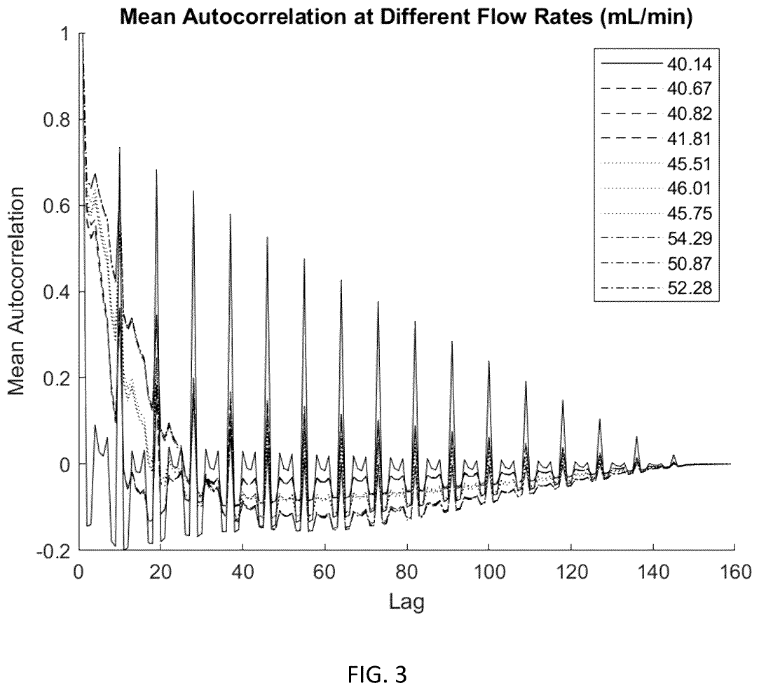

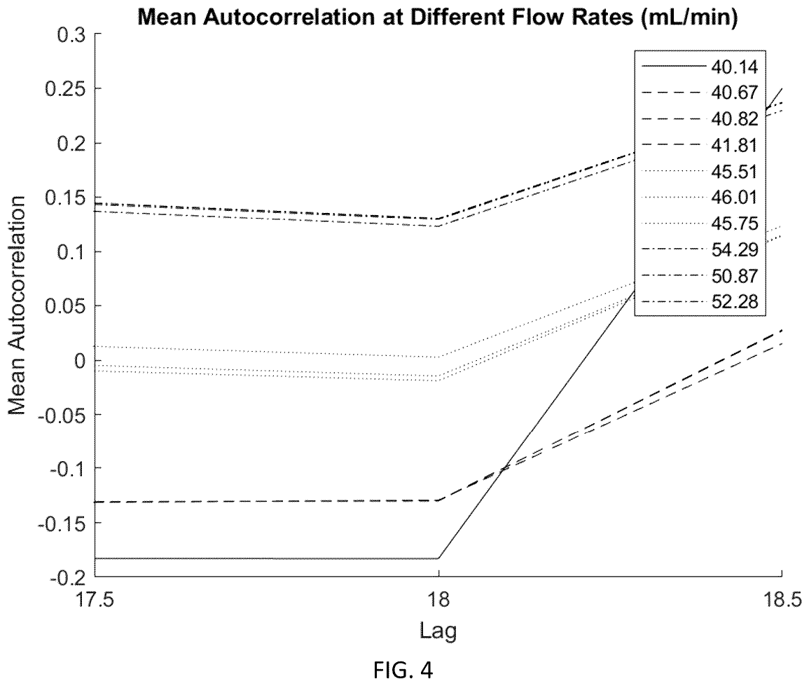

FIG. 3 is a graph 300 illustrating mean autocorrelation at different known flow rates (in mL/min) using near infrared spectroscopy at a given wavelength of light and at lag points of 0-160. In this exemplary data, the given wavelength used to generate the data in FIG. 3 was 1310 nm, but other IR wavelengths will generate similar patterns. FIG. 4 is a graph illustrating the mean autocorrelation at different known flow rates (in mL/min) using near infrared spectroscopy at a given wavelength of light (e.g., 1310 nm) over a lag range of 17.5-18.5. The mean autocorrelation of speckle patterns may be charted for different known flow rates, and this data may be used to determine unknown flow rates or flow velocities based on autocorrelations computed for speckle patters collected by system 100. The autocorrelation may be calculated at sequential lags or time intervals. A top lag may be selected by using a correlation coefficient to find the strongest linear relationship between the mean autocorrelation and the flowrates at every lag.

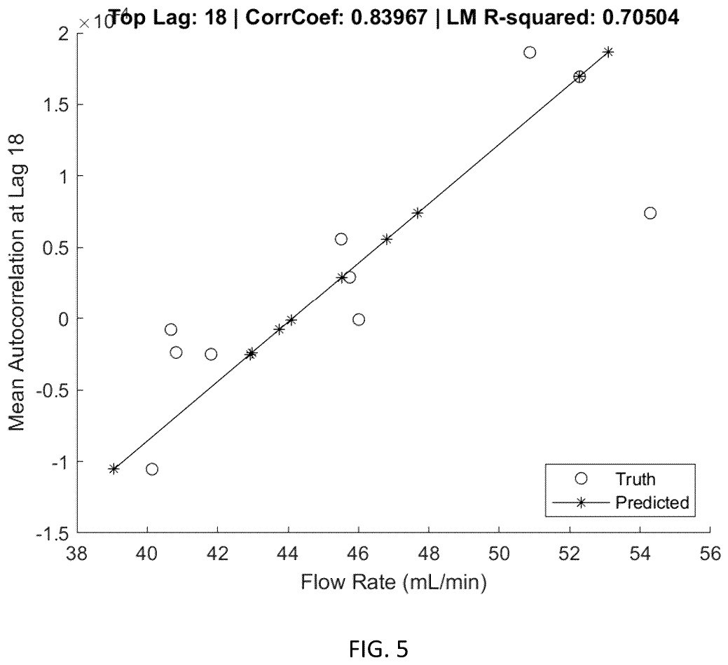

FIG. 5 is graph 500 illustrating a mean autocorrelation at a selected top lag for a given wavelength of light. The predicted mean autocorrelation at a particular lag (e.g., lag 18) at different flow rates is presented as a linear model (represented in FIG. 5 by asterisks connected by a line). The linear model may later be used in process 200 to predict flow rate based on autocorrelation measured within the blood flow. True values computed from actual measurements are represented with circles that are not connected by the line.

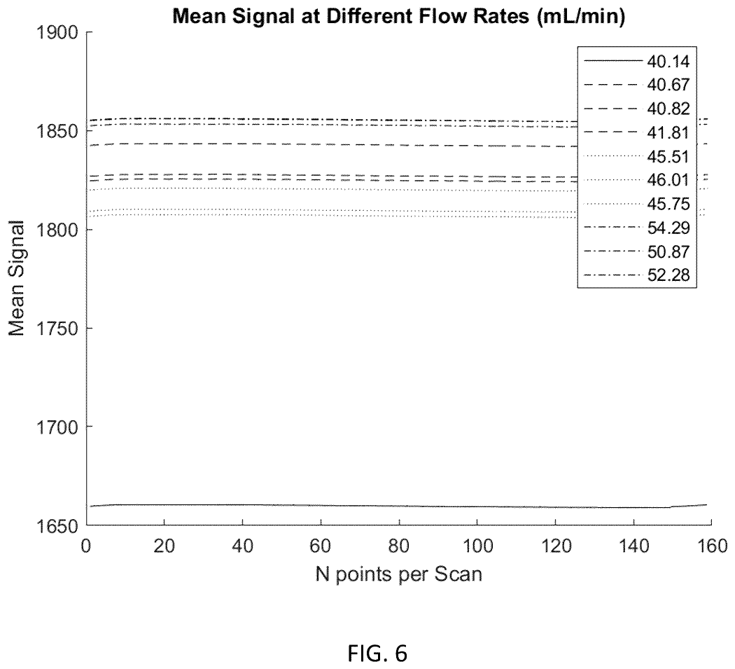

FIG. 6 is a graph of Mean Signal at Different Flow Rates and shows the averaged raw NIRS spectra across each count at each flow rate. Exploratory analysis to determine signal intensity anomalies potentially due to guidewires or other cardiovascular interventional instruments in the collection pathway could be gauged with this graph.

Referring back to step 208, the computing device 118 the flow rate is calculated (e.g., by computing device 208) by inputting the calculated autocorrelation and/or autocovariance at a particular lag into one or more linear models, e.g., linear models shown in FIGS. 5 and 7. Each linear model may be associated with a wavelength and a particular depth which is dictated by the wavelength of light used. The computing device 118 may select a linear model based on the first wavelength and the second wavelength of step 202. The output of the linear model may be a predicted flow rate at a particular distance from the catheter 102. In some embodiments, the output may include a predicted flow rate at a first depth associated with the first wavelength and a predicted flow rate at a second depth associated with the second flow rate.

At step 210, the computing device 118 may calculate FFR for blood vessel 100. As mentioned above, FFR is the ratio of maximum achievable blood flow through a blockage (e.g., an area of stenosis within the blood vessel) to the maximum achievable blood flow in the same vessel in the hypothetical absence of the blockage. To calculate FFR, the computing device 118 may obtain a hypothetical blood flow velocity for the blood vessel 100 if the plaque 112 was not present. In some embodiments, the hypothetical blood flow velocity may be obtained from a data storage of the computing device 118 or from another computing device (i.e., recorded and stored blood flow rates within healthy, unobstructed blood vessels). The computing device 118 may then calculate FFR using the blood flow velocity determined in step 208 and the hypothetical blood flow velocity.

At step 212, the computing device 118 may calculate the wall sheer stress for blood vessel 100. Wall sheer stress may be calculated using the blood flow velocity of step 208. Although step 212 follows step 210 in the flow chart of FIG. 2, it is not necessary to determine/compute the FFR before determining/computing the wall shear stress. In some embodiments, wall shear stress may calculated according to the following formula: Shear Stress=Blood Viscosity×Blood Velocity/Internal Diameter. More specifically, the formula for shear stress may be stated as:

τ=4μQ/πr³
Where:
τ represents the wall shear stress (in Pascals, Pa).
μ (mu) is the dynamic viscosity of blood (in pascal-seconds, Pa·s).
Q is the blood flow rate (in cubic meters per second, m³/s) (e.g., as determined by steps 202-208 of method 200).
r is the radius of the blood vessel (in meters, m) and can be found ultrasonically.

### Hardware and Software

Aspects of the subject matter disclosed herein may be implemented via control and computing hardware components, software (which may include firmware), data input components, and data output components. Hardware components include computing and control modules (e.g., system controller(s)), such as processing circuitry, configured to effect computational and/or control steps by receiving one or more input values, executing one or more algorithms stored on non-transitory machine-readable media (e.g., software) that provide instruction for manipulating or otherwise acting on or in response to the input values, and output one or more output values. Such processing circuitry may include one or more processors (e.g., one or more general purpose microprocessors and/or one or more other processors, such as one or more computer(s), an application specific integrated circuit (ASIC), field-programmable gate arrays (FPGAs), and the like), which processors may be co-located in a single housing or in a single data center or may be geographically distributed (i.e., the processing circuitry may be encompassed by a distributed computing apparatus). Such outputs may be displayed or otherwise indicated to a user for providing information to the user, for example information as to the status of the instrument or of a process being performed thereby, or such outputs may comprise inputs to other processes and/or control algorithms. Data input components comprise elements by which data is input for use by the control and computing hardware components. Such data inputs may comprise signals generated by sensors or scanners, such as, position sensors, speed sensors, accelerometers, environmental (e.g., temperature) sensors, motor encoders, barcode scanners, or RFID scanners, as well as manual input elements, such as keyboards, stylus-based input devices, touch screens, microphones, switches, manually-operated scanners, etc. Data inputs may further include data retrieved from memory. Data output components may comprise hard drives or other storage media, monitors, printers, indicator lights, or audible signal elements (e.g., chime, buzzer, horn, bell, etc.).

The above-described techniques can be implemented in digital and/or analog electronic circuitry, or in computer hardware, firmware, software, or in combinations of them. The implementation can be as a computer program product, i.e., a computer program tangibly embodied in a machine-readable storage device, for execution by, or to control the operation of, a data processing apparatus, e.g., a programmable processor, a computer, and/or multiple computers. A computer program can be written in any form of computer or programming language, including source code, compiled code, interpreted code, and/or machine code, and the computer program can be deployed in any form, including as a stand-alone program or as a subroutine, element, or other unit suitable for use in a computing environment. A computer program can be deployed to be executed on one computer or on multiple computers at one or more sites.

Method steps can be performed by one or more processors executing a computer program to perform functions of the invention by operating on input data and/or generating output data. Method steps can also be performed by, and an apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit). Subroutines can refer to portions of the computer program and/or the processor/special circuitry that implement one or more functions.

Processors suitable for the execution of a computer program include, by way of example, both general and special purpose microprocessors, and any one or more processors of any kind of digital or analog computer. Generally, a processor receives instructions and data from a read-only memory or a random access memory or both. The essential elements of a computer are a processor for executing instructions and one or more memory devices for storing instructions and/or data. Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Computer-readable storage devices suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

All possible combinations of elements and components described in the specification or recited in the claims are contemplated and considered to be part of this disclosure. It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the scope of the following appended claims.

1. A method for evaluating characteristics of a blood vessel and/or of blood flowing within the blood vessel, the method comprising:
(a) delivering light at one or more wavelengths into the blood vessel using a light delivery portion of a catheter;
(b) capturing reflected light at each of the one or more wavelengths and generating a signal corresponding to light captured at each of the one or more wavelengths using a light capturing portion of the catheter; and
(c) determining at least one of blood flow rate and blood flow velocity within the blood vessel from the signal generated in step (b) by the light capturing portion of the catheter.

2. The method of claim 1, further comprising:
(d) determining at least one of a wall sheer stress level and/or fractional flow reserve ratio for the blood vessel from the at least one of blood flow rate and blood flow velocity determined in step (c).

3. The method of claim 1, wherein the light delivery portion comprises a first optical guide extending along the catheter and the light capturing portion comprises a second optical guide extending along the catheter, and wherein the first optical guide is the same as or different than the second optical guide.

4. The method of claim 3, wherein the light delivery portion comprises an LED and/or laser coupled to the first optical guide and the light capturing portion comprises a photodetector coupled to the second optical guide.

5. The method of claim 1, wherein the light delivery portion comprises a light source located at an optical interface of the catheter, and the light capturing portion comprises a light detector located at the optical interface.

6. The method of claim 1, wherein step (c) comprises performing a time series analysis for the signal generated in step (b) by the light capturing portion of the catheter, wherein the time series analysis comprises an autocorrelation and/or autocovariance.

7. The method of claim 6, wherein the time series analysis is conducted for one or more lag times.

8. The method of claim 6, wherein step (c) comprises:
obtaining one or more linear models based on the time series analysis, wherein the one or more linear models provide an estimated flow rate at a selected lag time; and
inputting a result of the time series analysis for the captured reflected light at the selected lag time into the one or more linear models to determine a flow rate corresponding to the result of the time series analysis.

9. The method of claim 8, wherein each of the one or more linear models is associated with a different wavelength of light.

10. The method of claim 2, wherein step (d) comprises calculating the fraction flow reserve ratio by:
obtaining a hypothetical blood flow velocity within the blood vessel; and
comparing the calculated at least one of blood flow rate and blood flow velocity of the blood vessel with the hypothetical blood flow velocity.

11. The method of claim 2, wherein step (c) comprises determining the blood flow rate and step (d) comprises calculating the wall shear stress by the formula:
τ=4μQ/πr³,
where:
τ represents the wall shear stress;
μ (mu) is the dynamic viscosity of blood; and
Q is the blood flow rate determined in step (c).

12. The method of claim 1, wherein the one or more wavelengths include a first wavelength and a second wavelength, wherein the first wavelength is associated with a first penetration depth and the second wavelength is associated with a second penetration depth, the first penetration depth being less than the second penetration depth.

13. The method of claim 12, further comprising switching between the first wavelength and the second wavelength by activating a switch.

14. A system for evaluating characteristics of a blood vessel and/or of blood flowing within the blood vessel, the system comprising:
an intravascular catheter with a light delivery portion and a light capture portion; and
a controller coupled to each of the light delivery portion and the light capture portion of the catheter, wherein the controller is programmed to execute a procedure comprising:
(a) delivering light at one or more wavelengths into the blood vessel using the light delivery portion of the catheter;
(b) capturing reflected light at each of the one or more wavelengths and generating a signal corresponding to light captured at each of the one or more wavelengths using the light capturing portion of the catheter; and
(c) determining at least one of blood flow rate and blood flow velocity within the blood vessel from the signal generated in step (b) by the light capturing portion of the catheter.

15. The system of claim 1, wherein the procedure further comprises:
(d) determining at least one of a wall sheer stress level and/or fractional flow reserve ratio for the blood vessel from the at least one of blood flow rate and blood flow velocity determined in step (c).

16. The system of claim 14, wherein the light delivery portion comprises a first optical guide extending along the catheter and the light capturing portion comprises a second optical guide extending along the catheter, and wherein the first optical guide is the same as or different than the second optical guide.

17. The system of claim 16, wherein the light delivery portion comprises an LED and/or laser coupled to the first optical guide and the light capturing portion comprises a photodetector coupled to the second optical guide.

18. The system of claim 14, wherein the light delivery portion comprises a light source located at an optical interface of the catheter, and the light capturing portion comprises a light detector located at the optical interface.

19. The system of claim 14, wherein, to perform step (c), the controller is programed to perform a time series analysis for the signal generated in step (b) by the light capturing portion of the catheter, wherein the time series analysis comprises an autocorrelation and/or autocovariance.

20. The system of claim 19, wherein the time series analysis is conducted for one or more lag times.

21. The system of claim 19, wherein to perform step (c), the controller is programmed to obtain one or more linear models based on the time series analysis, wherein the one or more generated linear models provide an estimated flow rate at a selected lag time; and to input a result of the time series analysis for the captured reflected light at the selected lag time into the one or more linear models to determine a flow rate corresponding to the result of the correlation analysis.

22. The system of claim 21, wherein each of the one or more linear models is associated with a different wavelength of light.

23. The system of claim 15, wherein, to perform step (d), the controller is programmed to calculate the fraction flow reserve ratio by (1) obtaining a hypothetical blood flow velocity within the blood vessel; and (2) comparing the calculated at least one of blood flow rate and blood flow velocity blood flow velocity of the blood vessel with the hypothetical blood flow velocity.

24. The system of claim 12, wherein step (c) comprises determining the blood flow rate and step (d) comprises calculating the wall shear stress by the formula:
τ=4μQ/πr³,
where:
τ represents the wall shear stress;
μ (mu) is the dynamic viscosity of blood; and
Q is the blood flow rate determined in step (c).

25. The system of claim 14, wherein the one or more wavelengths include a first wavelength and a second wavelength, wherein the first wavelength is associated with a first penetration depth and the second wavelength is associated with a second penetration depth, the first penetration depth being less than the second penetration depth.

26. The system of claim 25, further comprising switching between the first wavelength and the second wavelength by activating a switch.
